(19) United States
(12) Patent Application Publication
(10) Pub. No.: US 2023/0056194 A1
(43) Pub. Date: Feb. 23, 2023

(54) STRESS ANALYSIS APPARATUS, STRESS ANALYSIS METHOD, AND COMPUTER-READABLE RECORDING MEDIUM

(71) Applicant: NEC Corporation, Minato-ku, Tokyo (JP)

(72) Inventor: Tasuku KITADE, Tokyo (JP)

(73) Assignee: NEC Corporation, Minato-ku, Tokyo (JP)

(21) Appl. No.: 17/797,617

(22) PCT Filed: Feb. 13, 2020
(86) PCT No.: PCT/JP2020/005638

§ 371 Date: Aug. 4, 2022

(51) Int. Cl. A61B 5/16; G16H 50/20; G16H 20/70; G16H 50/70

(52) U.S. Cl. CPC A61B 5/165; G16H 50/20; G16H 20/70; G16H 50/70

(57) ABSTRACT

A stress analysis apparatus includes: a stress factor extraction unit that extracts, from stress information in which a past state of stress of a user and information regarding a past activity of the user are associated with each other, a factor that increases stress in the user; a stress prediction unit that predicts an increase in stress in the user based on an activity schedule of the user and the extracted factor.

### TECHNICAL FIELD

The present invention relates to a stress analysis apparatus and a stress analysis method for analyzing stress on a person, and further relates to a computer-readable recording medium that includes a program recorded thereon for realizing the apparatus and method.

### BACKGROUND ART

People usually experience stress due to various external stimuli. In particular, such stress that occurs at work is called occupational stress and occurs due to work and human relationships in the workplace. Occupational stress often leads to development of mental illness such as depression, and consequently causes a decrease in productivity, employee departures, employee absences, and the like.

For this reason, it is necessary for companies to realize early detection of stress on employees, and attempts have been conventionally made to evaluate the state of a person who is subjected to stress. For example, Patent Document 1 discloses an apparatus for evaluating the state of the autonomic nervous function of the subject. The apparatus disclosed in Patent Document 1 first obtains biological information and the schedule of the subject, calculates indices indicating the state of stress from the biological information, and records the calculated indices and events included in the schedule in association with each other.

In this manner, according to the apparatus disclosed in Patent Document 1, the indices indicating the state of stress of the subject and the events the subject experienced are recorded in association with each other. Accordingly, by confirming the recorded content, events that cause the subject feel stress can be specified.

### LIST OF RELATED ART DOCUMENTS

### Patent Document

Patent Document 1: JP 2019-30389A

### SUMMARY OF INVENTION

### Problems to be Solved by the Invention

Although predicting how events which the subject has not experienced will influence the stress in the subject is most important, such prediction cannot be performed with the apparatus disclosed in the Patent Document 1. The apparatus disclosed in the Patent Document 1 merely records past events and indices at that time in association with each other.

An example object of the invention is to resolve the above problem and provide a stress analysis apparatus, a stress analysis method, and a computer-readable recording medium with which the future state of stress of the user can be predicted.

### Means for Solving the Problems

In order to achieve the above-described object, a stress analysis apparatus includes:

a stress factor extraction unit that extracts, from stress information in which a past state of stress of a user and information regarding a past activity of the user are associated with each other, a factor that increases stress in the user;

a stress prediction unit that predicts an increase in stress in the user based on an activity schedule of the user and the extracted factor.

In addition, in order to achieve the above-described object, a stress analysis method includes:

a factor extraction step of extracting, from stress information in which a past state of stress of a user and information regarding a past activity of the user are associated with each other, a factor that increases stress in the user; and

a stress increase prediction step of predicting an increase in stress in the user based on an activity schedule of the user and the extracted factor.

Furthermore, in order to achieve the above-described object, a computer readable recording medium according to an example aspect of the invention is a computer readable recording medium that includes recorded thereon a program,

the program including instructions that cause the computer to carry out

a factor extraction step of extracting, from stress information in which a past state of stress of a user and information regarding a past activity of the user are associated with each other, a factor that increases stress in the user; and

a stress increase prediction step of predicting an increase in stress in the user based on an activity schedule of the user and the extracted factor.

### Advantageous Effects of the Invention

As described above, according to the invention, it is possible to predict the future state of stress of the user.

### BRIEF DESCRIPTION OF THE DRAWINGS

FIG. 1 is a block diagram schematically illustrating a configuration of a stress analysis apparatus according to the example embodiment.

FIG. 2 is a block diagram specifically illustrating the configuration of the stress analysis apparatus according to the example embodiment.

FIG. 3 is a flowchart illustrating the operation at the time of construction of the stress model by the stress analysis apparatus according to the example embodiment.

FIG. 4 is a flowchart illustrating the operations at the time of stress factor extraction and stress prediction performed by the stress analysis apparatus according to the example embodiment.

FIG. 5 is a flowchart illustrating the operation at the time of generation of the advice performed by the stress analysis apparatus according to the example embodiment.

FIG. 6 is a block diagram illustrating an example of a computer that realizes the stress analysis apparatus according to the example embodiment.

### EXAMPLE EMBODIMENT

### Example Embodiment

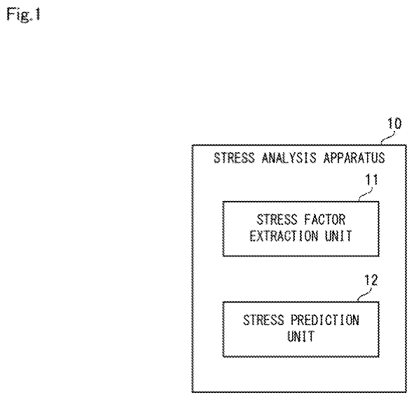

Hereinafter, a stress analysis apparatus, a stress analysis method, and a program according to an example embodiment of the invention will be described with reference to FIGS. 1 to 6.

[Apparatus Configuration]

First, a schematic configuration of the stress analysis apparatus according to the example embodiment will be described. FIG. 1 is a block diagram schematically illustrating a configuration of a stress analysis apparatus according to the example embodiment.

A stress analysis apparatus 10 according to the example embodiment illustrated in FIG. 1 is an apparatus for analyzing factors of stress in the user and predicting an increase in stress in the user in the future. As illustrated in FIG. 1, the stress analysis apparatus 10 is provided with a stress factor extraction unit 11 and a stress prediction unit 12.

The stress factor extraction unit 11 extracts a factor that increases stress in the user from stress information in which the past state of stress of the user and information about past activities of the user are associated with each other. The stress prediction unit 12 predicts an increase in stress in the user, based on the schedule of activities of the user and the extracted factor.

In this manner, the stress analysis apparatus 10 according to the example embodiment can extract a factor that may stress the user from the past information, and thus the state of stress on the user in the future can be predicted using the factor.

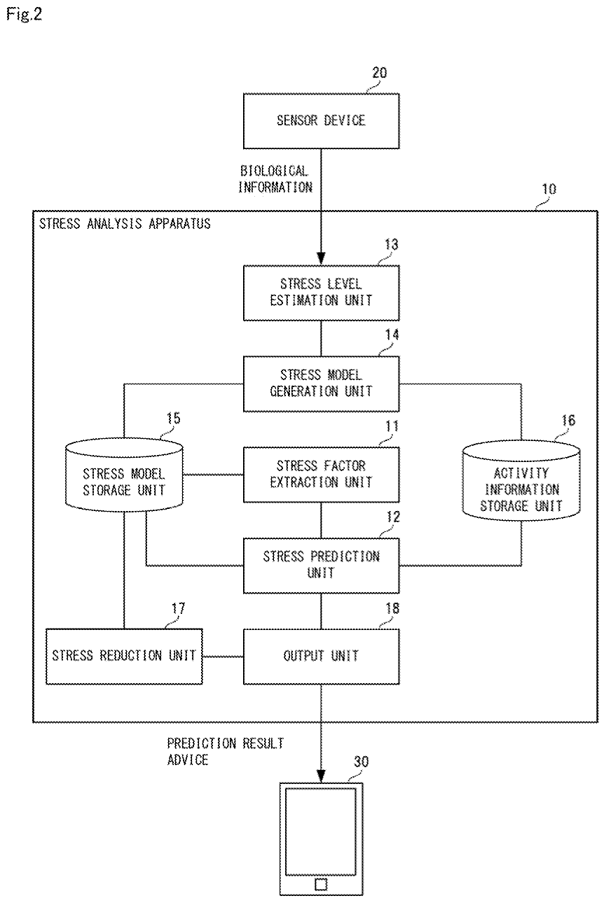

Next, the function and configuration of the stress analysis apparatus according to the example embodiment will be described in detail using FIG. 2. FIG. 2 is a block diagram specifically illustrating the configuration of the stress analysis apparatus according to the example embodiment.

As illustrated in FIG. 2, in the example embodiment, the stress analysis apparatus 10 is provided with a stress level estimation unit 13, a stress model generation unit 14, a stress model storage unit 15, an activity information storage unit 16, a stress reduction unit 17, and an output unit 18 in addition to the above stress factor extraction unit 11 and the stress prediction unit 12. Further, the stress analysis apparatus 10 is connected to a sensor device 20 that obtains the user's biological information and a terminal device 30 of the user via wired communication or wireless communication.

Also, the stress analysis apparatus 10 can also be constructed inside the terminal device 30 by a program according to the example embodiment, which will be described later, being installed in a computer of the terminal device 30 and executed. Examples of the terminal device 30 include a smartphone, a tablet-type terminal, and a laptop PC (Personal Computer).

The sensor device 20 is a device provided with a sensor that can detect biological information, and outputs the detected biological information. Examples of the sensor device 20 include a pulse rate meter for detecting heart rate, a skin electrometer for detecting the amount of skin electrical activity, a sweat meter for measuring the amount of sweat, an acceleration meter for detecting the acceleration of movement of a person. The sensor device 20 may also be a camera for capturing an image of the user's face.

The stress level estimation unit 13 obtains the biological information that is output from the sensor device 20, and estimates the stress level of the user from the obtained biological information. Specifically, for example, if the sensor device 20 is an acceleration meter, the stress level estimation unit 13 obtains an acceleration indicating the movement of the body of the user as the biological information. Then, the stress level estimation unit 13 estimates the stress level from the obtained acceleration according to technique disclosed in Reference Documents 1 or 2, which will be described below.

Reference Document 1: A. Sano et al., “Recognizing academic performance, sleep quality, stress level, and mental health using personality traits, wearable sensors and mobile phones,” in Wearable and Implantable Body Sensor Networks (BSN), 2015 IEEE 12th International Conference on, 2015, pp. 1-6

Reference Document 2: Yoshiki Nakashima et al., “Improvement in Chronic Stress Level Recognition by Using Both Full-term and Short-term Measurements of Physiological Features”, The 32nd Annual Conference of the Japanese Society for Artificial Intelligence, 2018.

If the sensor device 20 is a pulse rate meter, the stress level estimation unit 13 obtains the heart rate of the user as the biological information. If the sensor device 20 is a skin electrometer, the stress level estimation unit 13 obtains the amount of skin electrical activity of the user as the biological information. If the sensor device 20 is a sweat meter, the stress level estimation unit 13 obtains the amount of sweat as the biological information. And if the sensor device 20 is a camera, the stress level estimation unit 13 obtains a face image of the user as the biological information. Then, the stress level estimation unit 13 estimates the stress level using a method corresponding to the type of the obtained biological information, and outputs stress level information specifying the estimated stress level to the stress model generation unit 14.

Also, in the example embodiment, an existing method or a method which will be developed in the future can be used as the method for estimating the stress level from the acceleration, the heart rate, the amount of skin electrical activity, the amount of sweat, and the face image. Further, the stress level estimation unit 13 also estimates the change over time of the stress level by adding information indicating the time at which the biological information was output from the sensor device 20 to the estimated stress level.

In the example embodiment, the sensor device 20 may also be provided with a plurality of types of sensors and output a plurality of types of biological information in correspondence with the sensors. In this case, the stress level estimation unit 13 can also obtain the plurality of types of biological information and estimate the stress level using the plurality of types of biological information.

First, the stress model generation unit 14 receives the stress level information from the stress level estimation unit 13. Then, the stress model generation unit 14 specifies the information about the activity corresponding to the time of estimation of the stress level from the activity information storage unit 16. The activity information storage unit 16 stores information about activities of the user (activity information).

Examples of the activity information include information about meetings which the user attended (e.g., attendees, purpose, duration of the meeting), information about business trips which the user took (e.g., accompanying persons, purpose, destination, departure time), information about the tasks which the user worked on (e.g., content of tasks).

Next, the stress model generation unit 14 sets an activity variable Xᵢ according to a setting rule for each activity, using the specified activity information. The activity variable Xᵢ is composed of one or more values, and may be a binary value {0,1}, or a continuous value.

For example, if the activity is a meeting, the activity variable Xᵢ is composed of the number of attendees of the meeting Xᵢ, a binary flag (0 or 1) X₂ indicating whether a prescribed person attended, the number of attendees X₃ of a prescribed position or higher, the duration X₄ of the meeting, and the like. Note that if the duration of the activity such as the duration of the meeting is set to the activity variable, the value is a continuous value. At this time, the value may also be normalized.

Next, the stress model generation unit 14 constructs a learning model (hereinafter referred to as “stress model”) using the stress level Y estimated by the stress level estimation unit 13 and the activity variable Xᵢ set based on the activity information as training data. The constructed learning model is stored in the stress model storage unit 15. Not that, in this case, the answers to a questionnaire taken by the user, the results of judgments made by the user himself or herself and the like may also be used as the variable Y, in addition to the stress level estimated by the stress level estimation unit 13.

Specifically, the stress model generation unit 14 learns a weight coefficient aᵢ by performing multiple linear regression analysis using the stress level Y estimated by the stress level estimation unit 13 as the objective variable, and the activity variable Xᵢ as the explanatory variable. In this manner, the stress model indicated in Expression 1 is constructed. The constructed stress model is stored in the stress model storage unit 15.

Y=a₁X₁+a₂X₂+ . . . +aᵢXᵢ . . . +aₙXₙ (i=1,2,3, . . . ,n) Expression 1

In the example embodiment, the stress factor extraction unit 11 extracts the factor that increases stress in the user, using the stress model stored in the stress model storage unit 15 as the stress information. That is, the stress factor extraction unit 11 extracts the activity variable that is highly correlated with stress as the factor that increases stress in the user, from the stress model stored in the stress model storage unit 15.

Specifically, the stress factor extraction unit 11 specifies the activity variable Xᵢ with a high weight coefficient value aᵢ for each activity, and extracts the specified activity variable Xᵢ as the factor that increases stress in the user. Also, the stress factor extraction unit 11 outputs the extracted activity variable Xᵢ to the stress prediction unit 12.

In the example embodiment, the stress prediction unit 12 predicts an increase in stress in the user based on the activity variable Xᵢ that is output from the stress factor extraction unit 11 and the future activity information that is stored in the activity information storage unit 16.

Specifically, first, the stress prediction unit 12 sets the activity variable for each future activity using the future the activity information. Next, the stress prediction unit 12 compares the activity variable Xᵢ that is output with the set activity variable to specify the future activity including the activity variable Xᵢ that is output.

Next, the stress prediction unit 12 applies the activity variable that is set for the specified future activity to the stress model stored in the stress model storage unit 15 to calculate the stress level Y. Next, if the calculated stress level Y exceeds a threshold, the stress prediction unit 12 predicts an increase in stress in the user.

Also, if the stress level estimation unit 13 estimates the current stress level of the user, the stress prediction unit 12 calculates the difference between the calculated stress level Y and the current stress level. If the calculated stress level Y is larger than the current stress level and the difference is more than or equal to a threshold, the stress level estimation unit 13 also predicts an increase in stress in the user.

Further, if the future activity information stored in the activity information storage unit 16 is registered in units of time, for example, the stress level estimation unit 13 can also predict the time when stress in the user will increase.

The output unit 18 transmits the result of the prediction by the stress prediction unit 12 to the terminal device 30 of the user, for example. Upon receiving the prediction result, the terminal device 30 displays the received prediction result on a screen thereof. In this manner, the user can be aware of the likelihood of an increase in his or her stress. Note that, the transmission destination of the prediction result transmitted from the output unit 18 is not limited to the terminal device 30 of the user, and the transmission destination can include the terminal device of a stress manager, and a data management apparatus.

The stress reduction unit 17 extracts the factor that reduces stress in the user, and generates advice for reducing stress in the user based on the extracted factor. Also, the output unit 18 notifies the advice generated by the stress reduction unit 17 to the user.

Specifically, first, different from the stress factor extraction unit 11, the stress reduction unit 17 extracts the activity variable that is lowly correlated with stress as the factor that reduces stress in the user from the stress model stored in the stress model storage unit 15. For example, the stress reduction unit 17 specifies the activity variable having a low weight coefficient value aᵢ and extracts the specified activity variable as the factor that reduces stress in the user.

Next, the stress reduction unit 17 sets the activity variable for each future activity, using the future activity information. Next, the stress reduction unit 17 compares the activity variable that is set as the factor that reduces stress with the set activity variable, and specifies the future activity including the activity variable that is extracted as the factor that reduces stress.

Next, the stress reduction unit 17 calculates the stress level Y by applying the activity variable that is set for the specified future activity to the stress model stored in the stress model storage unit 15. If the calculated stress level Y is a threshold or less, the stress prediction unit 12 determines that stress in the user will be reduced.

Also, if the stress level estimation unit 13 estimates the current stress level of the user, the stress reduction unit 17 calculates the difference between the calculated stress level Y and the current stress level. If the calculated stress level Y is smaller than the current stress level and the difference is a threshold or more, the stress reduction unit 17 also determines that stress in the user will be reduced.

Further, if the future activity information stored in the activity information storage unit 16 is registered in units of time, for example, the stress reduction unit 17 can also predict the time when stress in the user will be reduced.

Then, if it is determined that the stress in the user will be reduced, the stress reduction unit 17 transmits, to the terminal device 30 of the user, the future activity specified in advance as advice for reducing the stress in the user, via the output unit 18. Upon receiving the advice, the terminal device 30 displays the received advice on the screen thereof. Accordingly, the user can be aware of the activity with which his or her stress can be reduced.

[Apparatus Operations]

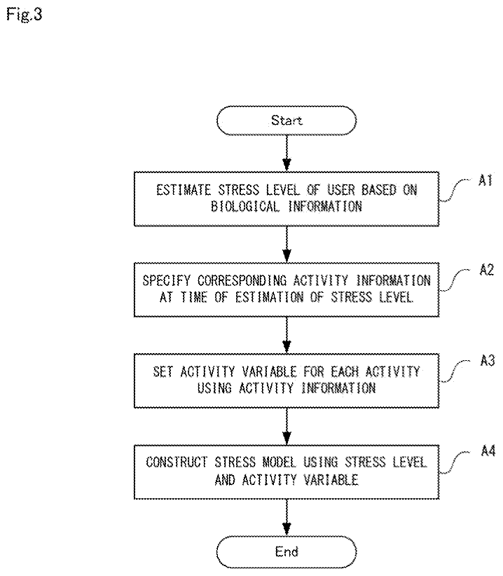

Next, the operations of the stress analysis apparatus 10 according to the example embodiment will be described using FIGS. 3 to 5. In the description below, FIGS. 1 and 2 are referred to as appropriate. In the example embodiment, the stress analysis method is implemented by operating the stress analysis apparatus 10. Therefore, the following description of the operations of the stress analysis apparatus 10 will be given in place of a description of the stress analysis method of the example embodiment.

The construction processing of a stress model will be described using FIG. 3. FIG. 3 is a flowchart illustrating the operation at the time of construction of the stress model by the stress analysis apparatus according to the example embodiment.

As illustrated in FIG. 3, first, the stress level estimation unit 13 estimates the stress level of the user based on the biological information that is output from the sensor device 20 during a set period or until an instruction is given by the user (step A1). The stress level estimation unit 13 also outputs the estimated stress level Y to the stress model generation unit 14 whenever estimation is performed.

Next, the stress model generation unit 14 accumulates the stress levels Y that are output, and when the period of the accumulated stress levels Y reaches a certain value, specifies the corresponding activity information at the time of estimation of the stress level Y from the activity information storage unit 16 (step A2).

Next, the stress model generation unit 14 sets the activity variable Xᵢ according to the setting rule for each activity using the activity information that is specified in step A2 (step A3).

After that, the stress model generation unit 14 constructs a stress model using the stress level Y estimated in step A1 and the corresponding activity variable Xᵢ set in step A3, as the training data (step A4). The constructed stress model is stored in the stress model storage unit 15.

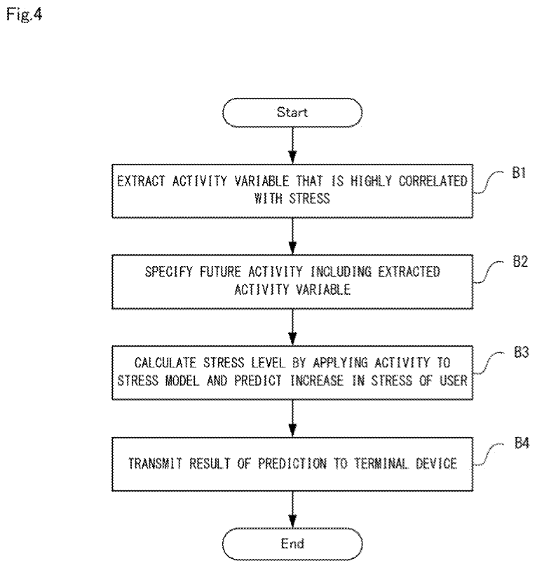

Stress factor extraction processing and stress prediction processing will now be described using FIG. 4. FIG. 4 is a flowchart illustrating the operations at the time of stress factor extraction and stress prediction performed by the stress analysis apparatus according to the example embodiment.

As illustrated in FIG. 4, in the example embodiment, first, the stress factor extraction unit 11 extracts the activity variable that is highly correlated with stress as the factor that increases stress in the user, from the stress model stored in the stress model storage unit 15 (step B1).

Next, the stress prediction unit 12 specifies, based on the activity variable that is set from the future activity information and the activity variable that is extracted in step B1, the future activity including the latter (step B2).

Next, the stress prediction unit 12 calculates the stress level by applying the activity specified in step B2 to the stress model constructed in step A4 illustrated in FIG. 3 and predicts an increase in stress of user using the calculated stress level (step B3).

After that, the output unit 18 transmits the result of prediction in step B3 to the terminal device 30 of the user (step B4). Upon receiving the result of prediction, in response to execution of step B3, the terminal device 30 displays the received result of prediction on the screen thereof. In this manner, the user can be aware of the likelihood of an increase in his or her stress.

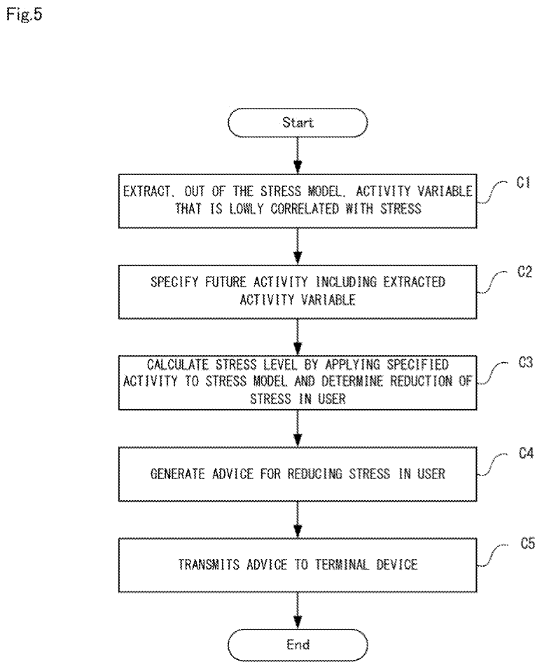

Stress reduction advice generation processing will now be described using FIG. 5. FIG. 5 is a flowchart illustrating the operation at the time of generation of the advice performed by the stress analysis apparatus according to the example embodiment.

As illustrated in FIG. 5, first, the stress reduction unit 17 extracts, out of the stress model stored in the stress model storage unit 15, the activity variable that is lowly correlated with stress, as the factor that reduces the stress in the user (step C1).

Next, the stress reduction unit 17 specifies, out of the activity variable that is set based on the future activity information and the activity variable that is extracted in step C1, the future activity including the latter (step C2).

Next, the stress reduction unit 17 calculates the stress level by applying the activity specified in step C2 to the stress model that is constructed in step A4 illustrated in FIG. 3, for each activity, and determines the reduction of the stress in the user using the calculated stress level (step C3).

Next, the stress reduction unit 17 sets the activity with which it is determined in step C3 that the stress in the user will be reduced, as the advice for reducing the stress in the user (step C4).

Next, the output unit 18 transmits the advice generated in step C4 to the terminal device 30 of the user (step C5). Upon receiving the advice, the terminal device 30 displays the received advice on the screen thereof. In this manner, the user can be aware of the activity with which his or her stress will be reduced.

### Effects of Example Embodiment

As described above, in the example embodiment, the stress analysis apparatus 10 can construct a stress model, and by using the stress model, a factor causing stress for the user can be extracted. Accordingly, the stress analysis apparatus 10 can predict a future state of stress of the user, and also generate advice for reducing stress.

[Program]

It suffices for a program in the example embodiment of the invention to be a program that causes a computer to carry out steps B1 to B4 illustrated in FIG. 4. Also, by this program being installed and executed in the computer, the stress analysis apparatus 10 and the stress analysis method according to the example embodiment can be realized. In this case, a processor of the computer functions and performs processing as the stress factor extraction unit 11 and the stress prediction unit 12.

Furthermore, it suffices for a program in the example embodiment of the invention to be a program that causes a computer to carry out steps A1 to A4 illustrated in FIG. 3 and steps C1 to C5 illustrated in FIG. 5. In this case, a processor of the computer functions and performs processing not only as the stress factor extraction unit 11 and the stress prediction unit 12, but also as the stress level estimation unit 13, the stress model generation unit 14, and stress reduction unit 17.

Also, the stress model storage unit 15 and the activity information storage unit 16 are realized by storing a data file constituting them into a storage device, such as a hard disk, included in the computer.

Furthermore, the program according to the present example embodiment may be executed by a computer system constructed with a plurality of computers. In this case, for example, each computer may function as one of the stress factor extraction unit 11 and the stress prediction unit 12, farther the stress level estimation unit 13, the stress model generation unit 14, and stress reduction unit 17. Also, the stress model storage unit 15 and the activity information storage unit 16 may be constructed on a computer that is different from the computer that executes the program according to the present embodiment.

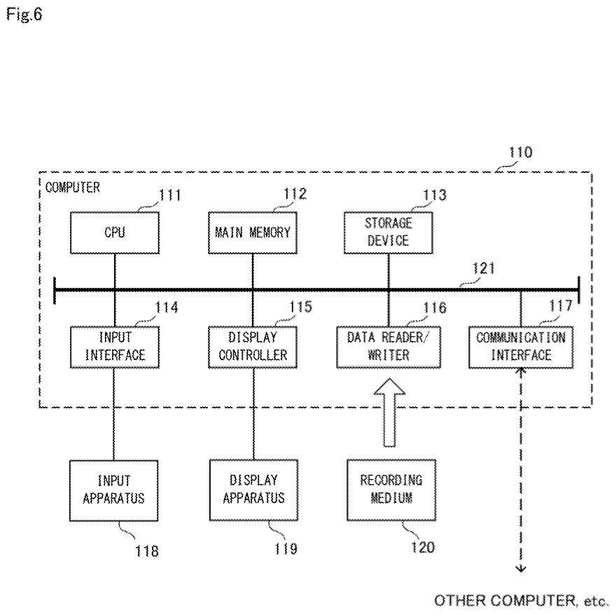

Using FIG. 6, the following describes a computer that realizes the stress analysis apparatus 10 by executing the program according to the present example embodiment. FIG. 6 is a block diagram illustrating an example of a computer that realizes the stress analysis apparatus according to the example embodiment.

As shown in FIG. 6, a computer 110 includes a CPU (Central Processing Unit) 111, a main memory 112, a storage device 113, an input interface 114, a display controller 115, a data reader/writer 116, and a communication interface 117. These components are connected in such a manner that they can perform data communication with one another via a bus 121. Note that the computer 110 may include a GPU (Graphics Processing Unit) or an FPGA (Field-Programmable Gate Array) in addition to the CPU 111, or in place of the CPU 111.

The CPU 111 carries out various types of calculation by deploying the program (codes) according to the present example embodiment stored in the storage device 113 to the main memory 112 and executing the codes in a predetermined order. The main memory 112 is typically a volatile storage device, such as a DRAM (dynamic random-access memory). Also, the program according to the present example embodiment is provided in a state where it is stored in a computer-readable recording medium 120. Note that the program according to the present example embodiment may be distributed over the Internet connected via the communication interface 117.

Also, specific examples of the storage device 113 include a hard disk drive and a semiconductor storage device, such as a flash memory. The input interface 114 mediates data transmission between the CPU 111 and an input apparatus 118, such as a keyboard and a mouse. The display controller 115 is connected to a display apparatus 119, and controls display on the display apparatus 119.

The data reader/writer 116 mediates data transmission between the CPU 111 and the recording medium 120, reads out the program from the recording medium 120, and writes the result of processing in the computer 110 to the recording medium 120. The communication interface 117 mediates data transmission between the CPU 111 and another computer.

Specific examples of the recording medium 120 include: a general-purpose semiconductor storage device, such as CF (CompactFlash®) and SD (Secure Digital); a magnetic recording medium, such as a flexible disk; and an optical recording medium, such as a CD-ROM (Compact Disk Read Only Memory).

Note that the stress analysis apparatus 10 according to the present example embodiment can also be realized by using items of hardware that respectively correspond to the components, rather than the computer in which the program is installed. Furthermore, a part of the stress analysis apparatus 10 may be realized by the program, and the remaining part of the stress analysis apparatus 10 may be realized by hardware.

A part or an entirety of the above-described example embodiment can be represented by (Supplementary Note 1) to (Supplementary Note 12) described below, but is not limited to the description below.

(Supplementary Note 1)

A stress analysis apparatus including:

a stress factor extraction unit configured to extract, from stress information in which a past state of stress of a user and information regarding a past activity of the user are associated with each other, a factor that increases stress in the user;

a stress prediction unit configured to predict an increase in stress in the user based on an activity schedule of the user and the extracted factor.

(Supplementary Note 2)

The stress analysis apparatus according to Supplementary Note 1, further comprising:

a stress reduction unit configured to extract a factor that reduces stress in the user from the stress information, generate advice for reducing stress in the user based on the extracted factor, and notify the generated advice to the user.

(Supplementary Note 3)

The stress analysis apparatus according to Supplementary Note 1 or 2,

wherein the stress factor extraction unit extracts a factor that increases stress in the user, using a learning model that is constructed by learning a relationship between the past state of stress of the user and the information regarding the past activity of the user, as the stress information.

(Supplementary Note 4)

The stress analysis apparatus according to Supplementary Note 3,

wherein the stress prediction unit predicts an increase in stress in the user by applying the activity schedule of the user and the extracted factor to the learning model.

(Supplementary Note 5)

A stress analysis method including:

a factor extraction step of extracting, from stress information in which a past state of stress of a user and information regarding a past activity of the user are associated with each other, a factor that increases stress in the user; and

a stress increase prediction step of predicting an increase in stress in the user based on an activity schedule of the user and the extracted factor.

(Supplementary Note 6)

The stress analysis method according to Supplementary Note 5, further including:

an advice notification step of extracting a factor that reduces stress in the user from the stress information, generating advice for reducing stress in the user based on the extracted factor, and notifying the generated advice to the user.

(Supplementary Note 7)

The stress analysis method according to Supplementary Note 5 or 6,

wherein, in the factor extraction step, factor that increases stress in the user is extracted, using a learning model that is constructed by learning a relationship between the past state of stress of the user and the information regarding the past activity of the user, as the stress information.

(Supplementary Note 8)

The stress analysis method according to Supplementary Note 7,

wherein, in the stress increase prediction step, an increase in stress in the user is predicted by applying the activity schedule of the user and the extracted factor to the learning model.

(Supplementary Note 9)

A computer readable recording medium that includes a program recorded thereon, the program including instructions that cause a computer to carry out:

a factor extraction step of extracting, from stress information in which a past state of stress of a user and information regarding a past activity of the user are associated with each other, a factor that increases stress in the user; and

a stress increase prediction step of predicting an increase in stress in the user based on an activity schedule of the user and the extracted factor.

(Supplementary Note 10)

The computer readable recording medium according to Supplementary Note 9, the program further including instructions that cause the computer to carry out:

an address notification step of extracting a factor that reduces stress in the user from the stress information, generating advice for reducing stress in the user based on the extracted factor, and notifying the generated advice to the user.

(Supplementary Note 11)

The computer readable recording medium according to Supplementary Note 9 or 10,

wherein, in the factor extraction step, a factor that increases stress in the user is extracted, using a learning model that is constructed by learning a relationship between the past state of stress of the user and the information regarding the past activity of the user, as the stress information.

(Supplementary Note 12)

The computer readable recording medium according to Supplementary Note 11,

wherein, in the stress increase prediction step, an increase in stress in the user is predicted by applying the activity schedule of the user and the extracted factor to the learning model.

Although the invention of the present application has been described above with reference to the example embodiment, the invention of the present application is not limited to the above-described example embodiment. Various changes that can be understood by a person skilled in the art within the scope of the invention of the present application can be made to the configuration and the details of the invention of the present application.

### INDUSTRIAL APPLICABILITY

As described above, according to the present invention, it is possible to predict the future state of stress of the user. The present invention is useful for human stress management system.

### REFERENCE SIGNS LIST

10 Stress analysis apparatus
11 Stress factor extraction unit
12 Stress prediction unit
13 Stress level estimation unit
14 Stress model generation unit
15 Stress model storage unit
16 Activity information storage unit
17 Stress reduction unit
18 Output unit
20 Sensor device
30 Terminal device
110 Computer
111 CPU
112 Main memory
113 Storage device
114 Input interface
115 Display controller
116 Data reader/writer
117 Communication interface
118 Input apparatus
119 Display apparatus
120 Recording medium
121 Bus

What is claimed is:

1. A stress analysis apparatus comprising:
at least one memory storing instructions; and
at least one processor configured to execute the instructions to:
extract, from stress information in which a past state of stress of a user and information regarding a past activity of the user are associated with each other, a factor that increases stress in the user;
predict an increase in stress in the user based on an activity schedule of the user and the extracted factor.

2. The stress analysis apparatus according to claim 1, further wherein further at least one processor configured to execute the instructions to:
extract a factor that reduces stress in the user from the stress information, generating advice for reducing stress in the user based on the extracted factor, and notifying the generated advice to the user.

3. The stress analysis apparatus according to claim 1,
wherein, further at least one processor configured to execute the instructions to: extract a factor that increases stress in the user, using a learning model that is constructed by learning a relationship between the past state of stress of the user and the information regarding the past activity of the user, as the stress information.

4. The stress analysis apparatus according to claim 3,
wherein, further at least one processor configured to execute the instructions to: predict an increase in stress in the user by applying the activity schedule of the user and the extracted factor to the learning model.

5. A stress analysis method comprising:
extracting, from stress information in which a past state of stress of a user and information regarding a past activity of the user are associated with each other, a factor that increases stress in the user; and
predicting an increase in stress in the user based on an activity schedule of the user and the extracted factor.

6. The stress analysis method according to claim 5, further comprising:
extracting a factor that reduces stress in the user from the stress information, generating advice for reducing stress in the user based on the extracted factor, and notifying the generated advice to the user.

7. The stress analysis method according to claim 5,
wherein, in the extraction of a factor, factor that increases stress in the user is extracted, using a learning model that is constructed by learning a relationship between the past state of stress of the user and the information regarding the past activity of the user, as the stress information.

8. The stress analysis method according to claim 7,
wherein, in the prediction of an increase in stress, an increase in stress in the user is predicted by applying the activity schedule of the user and the extracted factor to the learning model.

9. A non-transitory computer readable recording medium that includes a program recorded thereon, the program including instructions that cause a computer to carry out:
extracting, from stress information in which a past state of stress of a user and information regarding a past activity of the user are associated with each other, a factor that increases stress in the user; and
predicting an increase in stress in the user based on an activity schedule of the user and the extracted factor.

10. The non-transitory computer readable recording medium according to claim 9, the program further including instructions that cause the computer to carry out:
extracting a factor that reduces stress in the user from the stress information, generating advice for reducing stress in the user based on the extracted factor, and notifying the generated advice to the user.

11. The non-transitory computer readable recording medium according to claim 9,
wherein, in the extraction of a factor, a factor that increases stress in the user is extracted, using a learning model that is constructed by learning a relationship between the past state of stress of the user and the information regarding the past activity of the user, as the stress information.

12. The non-transitory computer readable recording medium according to claim 11,
wherein, in the prediction of an increase in stress, an increase in stress in the user is predicted by applying the activity schedule of the user and the extracted factor to the learning model.
